(19) United States
(12) Patent Application Publication
(10) Pub. No.: US 2023/0181745 A1
(43) Pub. Date: Jun. 15, 2023

(54) ZINC COMPLEXES AND THEIR USES

(71) Applicants: Demuris Limited, Newcastle Upon Tyne (GB); The Francis Crick Institute Limited, London (GB)

(72) Inventors: Richard Alexander Lewis, Newcastle Upon Tyne (GB); Jacqueline Hayles, London (GB); Paul Nurse, London (GB); Nicholas Edward Ellis Allenby, Newcastle Upon Tyne (GB); Jeffrey Errington, Embleton (GB)

(21) Appl. No.: 18/017,347

(22) PCT Filed: Jul. 20, 2021
(86) PCT No.: PCT/GB21/51861

§ 371 Date: Jan. 20, 2023

(30) Foreign Application Priority Data
Jul. 21, 2020 (GB) 2011239.7

(51) Int. Cl. A61K 47/54; A61P 35/02

(52) U.S. Cl. CPC A61K 47/547; A61P 35/02; A61K 47/545

(57) ABSTRACT

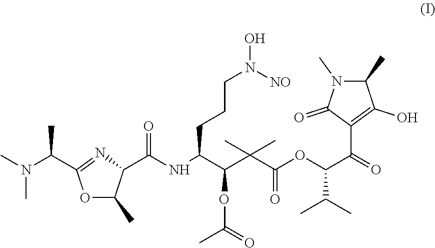

A zinc complex of a compound where the zinc complex is either a complex of JBIR-141 and a zinc ion and has the chemical formula C₃₁H₄₈N₆O₁₁Zn, or when the zinc complex is a complex of JBIR-142 and a zinc ion and has the chemical formula C₃₁H₄₈N₆O₁₂Zn.

The invention arose from a multidisciplinary research project in the fields of natural product chemistry and anticancer therapeutics.

### STATEMENTS OF INVENTION

According to a first aspect of the present invention is a zinc complex of a compound which has the chemical structure:

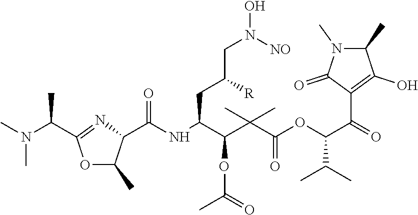

where R═H and the compound is known as JBIR-141, or where R═OH and the compound is known as JBIR-142, and the zinc complex is either a complex of JBIR-141 and a zinc ion and has the chemical formula is C₃₁H₄₈N₆O₁₁Zn, or is a complex of JBIR-142 and a zinc ion and has the chemical formula C₃₁H₄₈N₆O₁₂Zn.

According to a further aspect of the present invention is a zinc complex as described in for use as a medicament.

According to a further aspect of the present invention is a zinc complex as described in for use in the treatment of diseases associated with the overexpression of FoxO transcription factors.

According to a further aspect of the present invention is a zinc complex as described in for use in the treatment of diseases associated with the overexpression and predominantly nuclear localisation of FoxO transcription factors.

According to a further aspect of the present invention is a zinc complex as described in for use in the treatment of cancer associated with the overexpression of FoxO transcription factors.

According to a further aspect of the present invention is a zinc complex as described in for use in the treatment of cancer associated with the overexpression and predominantly nuclear localisation of FoxO transcription factors.

According to a further aspect of the present invention is a zinc complex as described in for use in the treatment of acute or chronic myeloid leukaemia.

According to a further aspect of the present invention is a pharmaceutical composition comprising a zinc complex as described in [002] or a pharmaceutically acceptable salt or solvate thereof, and one or more pharmaceutically acceptable excipients or carriers.

### Description of the Zinc Complex

The present invention is a zinc complex of a compound which has the chemical structure:

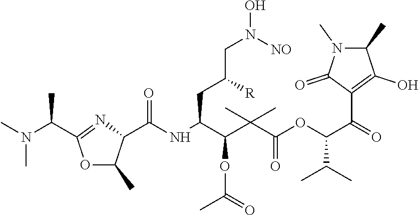

where R═H and the compound is known as JBIR-141, or where R═OH and the compound is known as JBIR-142, and the zinc complex is either a complex of JBIR-14I and a zinc ion and has the chemical formula is C₃₁H₄₈N₆O₁₁Zn, or is a complex of JBIR-142 and a zinc ion and has the chemical formula C₃₁H₄₈N₆O₁₂Zn.

JBIR-141 is a known molecule which has been published (Kawahara T, Kagaya N, Masuda Y, Doi T, Izumikawa M, Ohta K, Hirao A and Shin-ya K. 2015. Foxo3a inhibitors of microbial origin, JBIR-141 and JBIR-142. Organic Letters, 17(21): 5476-9). The structure of JBIR-141 is shown below.

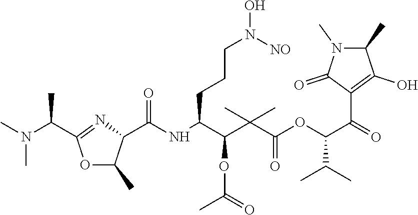

The zinc complexed form of JBIR-141, which is a novel chemical entity, and which is first described herein, is also known as “S149” and is herein often referred to as “S149”. The above structure of JBIR-141 is also the structure of S149, with the exception that two hydrogen atoms are substituted by a zinc ion (Zn²⁺) so that the chemical formula of JBIR-141 is C₃₁H₅₀N₆O₁₁and the chemical formula of S149/the zinc complex of JBIR-141 is C₃₁H₄₈N₆O₁₁Zn.

Kawahara et al., 2015 also describe a second molecule—JBIR-142 which is almost identical to JBIR-141 and differs from it only in that a hydrogen atom is substituted by a hydroxy group. The structure of JBIR-142 is as follows:

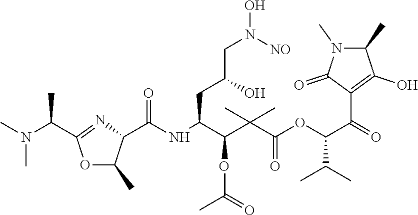

Although we have not produced the zinc complexed form of JBIR-142 in view of the fact that its published activity (Kawahara et al., 2015) is very similar to that of JBIR-141 and the difference in structure and chemical formula is a minor one we believe that the zinc complex of JBIR-142 possesses similar properties to that of S149 (the zinc complex of JBIR-141). Thus, the present invention also relates to, and encompasses, the zinc complex of JBIR-142. The chemical formula of JBIR-142 is C₃₁H₅₀N₆O₁₂ and the chemical formula of the zinc complex of JBIR-142 is C₃₁H₄₈N₆O₁₂Zn.

The journal paper published by Kawahara et al.,—“Foxo3a inhibitors of microbial origin, JBIR-141 and JBIR-142” makes no reference to either JBIR-141 or JBIR-142 (or any of their variants/derivatives/degradation products etc) as being able to, or being capable of binding Zn²⁺. Thus, S149, i.e. the zinc complexed form of JBIR-141 represents a novel entity and is not anticipated by the disclosure of Kawahara et al., 2015. Likewise, the zinc complexed form of JBIR-142 also represents a novel entity and is similarly not anticipated by the disclosure of Kawahara et al., 2015.

Zinc complexes of the present invention may be produced by synthetic chemical means. However, preferably, they may also be extracted from material produced by culturing bacteria which are capable of producing the complexes. Such bacteria include DEM21859 (described herein in paragraphs [043]-[048]) but may also include other strains of Streptomyces coeruleofuscus. Other suitable species of bacteria capable of being used to produce zinc complexes of the present invention are those described in Kawahara et al., 2015 which are capable of producing JBIR-141 and/or JBIR-142, which may be purified as described in Kawahara et al., 2015 before being complexed with zinc to yield the zinc complexes of the present invention.

Compounds that have the same molecular formula but differ in the nature or sequence of bonding of their atoms or the arrangement of their atoms in space am termed “Isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The zinc complex as described in [002] may possess one or more asymmetric centres; such compounds can therefore be produced as individual (R)- or (S)-stereoisomers or as mixtures thereof. Unless indicated otherwise, the description or naming of a particular complex in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. The zinc complex as described in [002] may have geometric isomeric centres (E- and Z- isomers). It is to be understood that the present invention encompasses all optical, diastereoisomers and geometric isomers and mixtures thereof that possess anti—foxO transcription factor activity.

The present invention also encompasses a zinc complex as described in [002] which comprise one or more isotopic substitutions. For example, —H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain zinc complexes as described in [002] may exist in solvated as well as unsolvated forms such as, for example, hydrated forms. It is to be understood that the invention encompasses all such solvated forms that possess inhibitory activity against foxo transcription factors. It is also to be understood that certain a zinc complex as described in may exhibit polymorphism, and that the invention encompasses all such forms that possess inhibitory activity towards FoxO transcription factors.

A zinc complex as described in [002] may exist in a number of different tautomeric forms and references to a zinc complex as described in [002] include all such forms. For the avoidance of doubt, where a zinc complex can exist in one of several tautomeric forms, and only one is specifically described or shown, all others are nevertheless embraced by the structural formula provided in [002]. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

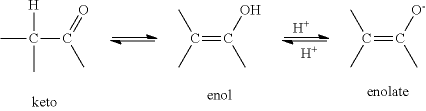

### The Use of Zinc Complexes of the Invention in Medical Treatments.

The zinc complexes of the invention are to be used to treat medical conditions mediated by forkhead box (FOX) transcription factors and particularly FoxO transcription factors. According to a further aspect of the present invention is a zinc complex as described in [002] for use in the treatment of diseases associated with the overexpression of FoxO transcription factors. According to a further aspect of the present invention is a zinc complex as described in [002] for use in the treatment of diseases associated with the overexpression and predominantly nuclear localisation of FoxO transcription factors. According to a further aspect of the present invention is a zinc complex as described in [002] for use in the treatment of cancer associated with the overexpression of FoxO transcription factors. According to a further aspect of the present invention is a zinc complex as described in [002] for use in the treatment of cancer associated with the overexpression and predominantly nuclear localisation of FoxO transcription factors. According to a further aspect of the present invention is a zinc complex as described in [002] for use in the treatment of acute or chronic myeloid leukaemia.

FoxO Transcription Factors and their Role in Maintenance of Cancers.

The forkhead box (FOX) protein family consists of 19 sub-families of transcription factors which share a highly conserved DNA-binding domain of ˜110 amino acids, the forkhead box domain (also known as the winged-helix domain). The 0 sub-group (FOXO or FoxO or foxo) contains four members—FOXO1, FOXO, 3, FOXO4 and FOXO6. The first three of these are ubiquitously expressed, the level depending on the tissue, whereas FOXO6 is only expressed in the central nervous system.

The expression and activity of FOXO factors are controlled by post-translational modifications. For example, a major mechanism of their regulation is by phosphorylation by AKT on three residues (T32, S253 and S315 of FOXO3) following growth factor stimulation. The phosphorylations allow the export of the FOXO factors from the nucleus (where they are nuclear transcription factors active in inducing gene expression) to the cytoplasm (where effectively they are sequestered and inactive). Thus, the level of FOXO factor expression and their sub-cellular localisation must be evaluated when assessing FOXO factor activity. FOXO factors have been shown to be involved in many human disease states (Maiese K, Chong Z Z, and Shang Y C. 2008. OutFOXing disease and disability: the therapeutic potential of targeting FoxO proteins. Trends in Molecular Medicine 14(5): 217-227) and in particular cancer (Yang J-Y, and Hung M-C. 2009. A new fork for clinical application: targeting forkhead transcription factors in cancer. Clinical Cancer Research, 15(3): 752-757), Coomans de Branckne A, and Demoulin J-B. 2016. Foxo transcription factors in cancer development and therapy. Cellular and Molecular Life Sciences, 73(6):1159-72.

High level of FOXO3a expression has been shown to be associated with poor prognosis in Acute Myeloid Leukaemia (AML). For example, when 43 patients with high FOXO3a expression and 49 with low FOXO3a RNA levels were evaluated for relapse free survival (RFS) the RFS was higher for the former group (22 out of 43: 51.2%) than the latter (17 out of 49: 34.7%). Therefore, the RFS at 2 years was significantly shorter in the high FOXO3a group than in the low FOXO3a group (50% vs 66%). (Santamaria C M, Chillon M C, Garcia-Sanz R, Perez C, Caballero M D, Ramos F, Garcia de Coca A, Alonso J M, Giraldo P, Bernal T, et al., 2009. High FOXO3a expression is associated with a poorer prognosis in AML with normal cytogenetics. Leukemia Research, 33: 1706-1709). Whilst Santamaria et al., did not explain the mechanism by which FOXO3a supports AML the later study of Naka et al., (Naka K, Hoshii T, Muraguchi T, Todokoro Y, Ooshio T, Kondo Y, Nakao S, Motoyama N and Hirao A. 2010. TGF-β—FOXO signalling maintains leukaemia-initiating cells in chronic myeloid leukaemia. Nature, 463(7281): 676-680) investigated the role of FOXO3a in maintenance of LIC's (Leukaemia Initiating Cells) responsible for Chronic Myeloid Leukaemia (CML). The results of serial bone marrow transplants (BMT's) of Fox03a^{+/+} and Fox03a^{-/-}LIC's indicated that FOXO3a is essential for the long-term maintenance of leukaemia initiating potential. Also, apoptotic cells were increased in Fox03a^{-/-} CML-affected mice compared to controls and the fact that Annexin-V⁺ and TUNEL⁺ cells were more frequent among Fox03a^{-/-} than Fox03a^{+/+}LIC's indicated that Foxo3a is required for LIC survival because it mediates suppression of apoptosis (Naka K, Hoshii T, Muraguchi T, Todokoro Y, Ooshio T, Kondo Y, Nakao S, Motoyama N and Hirao A. 2010. TGF-β-FOXO signalling maintains leukaemia-initiating cells in chronic myeloid leukaemia. Nature, 463(7281): 676-680. The importance in FoxO factors in maintenance of AML LICs has been confirmed by other investigators (Sykes S M, Lane S W, Bullinger L, Kalaitzidis D, Yusuf R, Saez B, Ferraro F, Mercier F, Singh H, Brumme K M et al., 2011. AKT/FOXO signalling enforces reversible differentiation blockade in myeloid leukemias. Cell, 146(5):697-708). Unsurprisingly, attempts have been made to inhibit CML stem cells by reducing FoxO activity. For example, Naka et al., 2010 describe using Ly364947 (an inhibitor of TGF-β signalling, effectively activating AKT and so suppressing FoxO factor activity) in combination with imatinib to reduce CML stem cell frequency (Naka K, Hoshii T, and Hirao A. 2010. Novel therapeutic approach to eradicate tyrosine kinase inhibitor resistant chronic myeloid leukemia stem cells. Cancer Science, 101(7): 1577-1581.

Kawahara et al., show that JBIR-141 & JBIR-142 possess the ability to inhibit the transcriptional activity of the forkhead transcription factor, Foxo3a and herein we describe the anti-proliferative effect of S149 against a range of leukaemia cell lines—see paragraphs [068]-in the “Embodiment of the invention”.

We have also tested the zinc-free form of S149 (which is identical to JBIR-141) and the zinc complexed form (S149) in parallel against the unicellular yeast Schizosaccharomyces pombe in bioassays see paragraph [054] of the “Embodiment of the invention”. Significantly, we have shown that the zinc-free form of S149 (which is identical to JBIR 141) is not as bioactive as the zinc complexed form (S149)—see paragraph [054] of the “Embodiment of the invention” herein. Prima facie, this shows that the zinc complexed form of JBIR-141, i.e. S149, has improved bioactivity over JBIR-141 and this represents a real advance over the prior art.

### The Use of Zinc Complexes of the Invention in Preparing Pharmaceuticals.

In one aspect of the present invention there is provided a pharmaceutical composition which comprises a zinc complex as described in [002], or a pharmaceutically acceptable salt, hydrate or solvate thereof, in association with a pharmaceutically acceptable diluent or carrier.

The compositions of the invention may be in a form suitable for oral use (for example, as tablets, lozenges, hard or soft capsules, aqueous or oily suspensions, emulsions, dispersible powders or granules, syrups or elixirs), for topical use (for example, as creams, ointments, gels, or aqueous or oily solutions or suspensions), for administration by inhalation (for example, as a finely divided powder or a liquid aerosol), for administration by insufflation (for example, as a finely divided powder) or for parenteral administration (for example, as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a zinc complex of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from I to 30 mg) compounded with an appropriate and convenient amount of excipients which may vary from about 5 to about 98 percent by weight of the total composition.

The size of the dose for therapeutic or prophylactic purposes of a zinc complex of the invention will naturally vary according to the nature and severity of the conditions, the age and sex of the animal or patient and the route of administration, according to well-known principles of medicine.

In using a zinc complex of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Routes of Administration

The zinc complex as described in [002] or pharmaceutical compositions comprising this zinc complex may be administered to a subject by any convenient mute of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufilation therapy using, e.g., via an aerosol, e.g., through the mouth or nose); rectal (e.g., by suppository or enema); vaginal (e.g., by pessary); Parenteral, for t, by injection, including subcutaneous, intradertnal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Assessment of the Prior Art

Results of Literature Search for Molecules Similar in Structure to that of the Present Invention

A search of the scientific literature for molecules similar in structure to that of S149 was performed. Excepting 1131R-141 and JBIR-142, which we have already described and which have been published in Kawahara et al, 2015, the most similar prior art molecule which we have identified is known as “mycobactin”. This is a siderophore which binds iron and has been well studied (Fang Z, Sampson S L, Warren R M, Gey van Pittius N C and Newton-Foot M. 2015. Iron acquisition strategies in mycobacteria. Tuberculosis, 95(2):123-130; Quadr L E N, Sello J, Keating T A, Weinreb P H, Walsh C T. 1998. Identification of a Mycobacterium tuberculosis gene cluster encoding the biosynthetic enzymes for assembly of the virulence-conferring siderophore mycobactin. Chemistry & Biology, 5: 631-645). Mycobactin's structure is analogous, rather than similar to that of S149, but resembles it in that the centrally located, derivatised amino acid residue of both molecules is a basic one (in the case of myobactin, lysine, and in the case of S149, ornithine). Additionally, the modified amino acid residue with a cyclised side-chain located adjacent (N-terminally located) to the modified basic amino-acid residue is a hydroxylated one in both molecules (serine in the case of mycobactin and threonine in the case of S149). Furthermore, both molecules possess terminally located ring structures derived from amino acids, in the case of mycobactin a seven membered ring derived from lysine and in the case of S149 a tetramic acid moiety derived from alanine.

However, it should be noted that the structure of mycobactin differs significantly from that of S149 and does not teach towards it, i.e. no one would be inspired by the documentation relating to mycobactin to identify or synthesise S149. It is also worth noting that mycobactin is a siderophore (iron binding molecule) as opposed to a zincophore (S149) and that mycobactin has not been shown to have an inhibitory effect on Foxo transcription factors, or to possess anticancer activity.

### Results of Literature Search for Natural Product Molecules Capable of Binding Zinc

A literature search for bacterially derived natural product molecules capable of binding zinc (“zincophores”) was performed. Compared to the number of iron binding natural products (siderophores) zincophores (reviewed in Johnstone T C, and Nolan E. M. 2015. Beyond iron: non classical biologicalfimctions of bacterial siderophores. Dalton Transaction, 44(14): 6320-6339) are relatively few in number and include, coelibactin (Kallifidas D, Pascoe B, Owen G A, Strain-Damerell C M, Hong H J, Paget M S. 2010. The zinc-responsive regulator Zur controls expression of the coelibactin gene cluster in Streptomyces coelicolor. Journal of Bacteriology, 192(2): 608-11), transvalencin (Hoshino Y, Mulcai A, Yazawa K, Uno J, Ando A, Mikami Y, Fukai T, Ishikawa J, Yamaguchi K. 2004. Transvalencin A, a thiazolidine zinc complex antibiotic produced by a clinical isolate of Nocardia transvalensis. II. Structure elucidation. Journal of Antibiotics (Tokyo) 57(12): 803-7), micacocidin (Kobayashi S, Hidaka S, Kawamura Y, Ozaki M, Hayase Y. 1998. Micacocidin A, B and C, novel antimycoplasma agents from Pseudomonas.sp. I. Taxonomy, fermentation, isolation, physico-chemical properties and biological activities. Journal of Antibiotics (Tokyo) 51(3): 323-7), pyochelin (Brandel J, Humbert N, Elhabiri M, Schalk I J, Mislin G L, Albrecht-Gary A M. 2012. Pyochelin, a siderophore of Pseudomonas aeruginasa: physicochemical characterization of the iron (III), copper (II) and zinc (II) complexes. Dalton Transactions. 41(9):2820-34), yersiniabactin (Bobrov A G, Kirillina O, Fetherston J D, Miller M C, Burlison J A, Perry R D. 2014. The Yersinia pestis siderophore, yersiniabactin, and the ZnuABC system both contribute to zinc acquisition and the development of lethal septicaemic plague in mice. Molecular Microbiology, 93(4):759-75) and tetrazolemycin (Liu N, Shang F, Xi L, Huang Y. 2013. Tetroazolemycins A and B, two new oxazole-thiazole siderophores from deep-sea Streptomyces olivaceus FXJ8.012. Marine Drugs, 11(5): 1524-33). These known zincophores possess structural motifs in common i.e. thiazolidine and thiazoline rings and salicylic acid moieties which are not present in S149 and JBIR-141. Therefore, it is not obvious from the molecular structure that JBIR-141 would be able to bind a zinc ion and so form the zinc complex S149, as structural elements known to be associated with zincophores and published in the scientific literature are not present. Another zinc-binding molecule described in the literature is zincphyrin (Toriya M, Yaginuma S, Murofushi S, Ogawa K, Muto N, Hayashi M, Matsumoto K. 1993. Zincphyrin, a novel coproporphyrin III with zinc from Sireptomyces sp. The Journal of Antibiotics (Tokyo), 46(1): 196-200). However, the structure of S149 does not resemble the structure of zincphyrin which is a porphyrin type molecule.

The structures of several natural product molecules are known which possess a nitroso group thought to be involved in metal ion chelation/complex formation. However, although JBIR-141 and JBIR-142 possess a nitroso group the prior art would suggest that they would be likely to form Cu²⁺ complexes rather than Zn²⁺ complexes, as cupferron (Heyn A H, Dave N G. 1966. Precipitation of metal-cupferron complexes from homogeneous solution-I: determination of copper. Talanta. 13(1): 27-32), dopastin (Shiino M, Watanabe Y, Umezawa K. 2001. Synthesis of N-substituted N-nitrosohydroxylamines as inhibitors, of mushroom lyrosinase. Bioorganic and Medicinal Chemistry, (5): 1233-40), and neocupferron all bind Cu²⁺ and not Zn²⁺.

### Results of Literature Search for FoxO Inhibitors

The involvement of FoxO transcription factors in many disease states has unsurprisingly led to it being a therapeutic target. We have done searches of the patent and scientific literature focusing on FoxO inhibitors and particularly those relating to treatment of leukaemia and especially those relating to acute and/or chronic myeloid leukaemia to determine the closest prior art to the present invention and have concluded that the abovementioned document Kawahara T, Kagaya N, Masuda Y, Doi T, Izurnikawa M, Ohta K, Hirao A and Shin-ya K. 2015. Foxo3a inhibitors of microbial origin, JBIR-141 and JIM-142. Organic Letters, 17(21): 5476-5499, represents the closest prior art.

Other documents identified during the search include, US2014227289 (COHEN ET A L.,) which describes treating insulin resistance by administering an inhibitor (suitable oligonucleotide based, antibody or small organic molecules are listed) of PERK (protein kinase RNA-like endoplasmic reticulum kinase) which leads to a reduction of Foxo transcription factor activity. WO2020037326 (Al) (FREQUENCY THERAPUETICS INC) describes the use of a FoxO inhibitor (AS 184285) in treating sensorineural hearing loss by increasing proliferation of Lgr5+ cochlear cells. AU2017239562 (Al) (ICAHN SCHOOL OF MEDICINE AT MOUNT SINAI) describes tricyclic chemical modulators of FoxO transcription factor activity and their use as anticancer agents. US2014206624 (Al) (SYKES ET AL.,) describes compositions and methods for the treatment of leukaemia by inhibiting Foxo transcription factors. The use of LOM612, a newly synthesised isothiazolonaphthoquinone as a FoxO relocator which exerts an antiproliferative effect on human cancer cell lines has also been described (Cautain B, Castillo F, Musso L, Ferreira B I, de Pedro N, Rodriguez Quesada L, Machado S, Vicente F, Dallavalle S, Link W. 2016. Discovery of a novel isothiazolonaphthoquinone-based small molecule activator of FOXO nuclear-cytoplasmic shuttling. PLoS One, 11(12): e0167491. doi: 10.1371/journal.pone.0167491). Although these documents describe inhibitors of FoxO transcription factors in medical conditions, including cancer, none of the inhibitors which they describe are identical to, or similar to those of the present invention, nor do they teach towards them.

### EMBODIMENT OF THE INVENTION

Growth of the Actinomycete Strain which Produces the Zinc Complex of the invention

The first embodiment of the invention is a zinc complexed form of the molecule known as JB1R-141. JBIR-141 has the chemical formula C₃₁H₅₀N₆O₁₁. This particular zinc complex, which has the chemical formula C₃₁H₄₈N₆O₁₁Zn, is also known as “S149” by which is often referred to herein. S149 is produced by an actinomycete strain known by its Dernuris code number—DEM21859.

The DEM21859 strain was identified as being of interest during a screen of a collection of actinomycete bacterial strains which produced antifungal agents. The screen was intended to identify compounds which induced morphological changes/non wild-type cell size/shape phenotypes in the fission yeast Schizosaccharomyces pombe. This approach was intended to identify compounds which target the cell cycle mechanism, and which presumably have effects on cell division and/or cell size and shape. When plugs cut from a confluent lawn of cultured DEM21859 were bioassayed by being placed on a lawn of S. pombe cells it was found that in addition to producing a zone of inhibition in the S. pombe lawn the pombe cells within the zone exhibited a phenotype described as “small and round”. S. pombe normally grows as a sausage shaped rod during exponential growth, therefore, the usually small and spherical shaped cells were of interest.

The results of the screen have already been published (Lewis R A, Li J, Allenby N E E, Errington J, Hayles J and Nurse P. 2017. Screening and purification of natural products from actinomycetes that affect the cell shape offission yeast. Journal of Cell Science, 130: 3173-3185). DEM21859 was not disclosed/included in the list of strains screened and is not mentioned in the paper or the supplementary information.

DEM21859 forms green/grey spores which take ˜0.7 days to form when cultured on GYM agar plates. The spores are spiky/prickly when viewed using the scanning electron microscope. It forms dark brown pigment when cultured on certain media for long periods (>7 days). The 16S rRNA sequence was determined for DEM21859. The closest BLAST match was Streptomyces coeruleofuscus with no mismatches out of 1413 nucleotides of sequence. The physical characteristics of DEM21859 are consistent with the published description of Streptomyces coeruleofuscus (Trejo W H, and Bennett R E. 1963. Streptomyces species comprising the blue-spore series. Journal of Bacteriology, 85: 676-90). Only one publication mentions antimicrobial properties of Streptomyces coeruleofuscus and this is as a treatment for plant root pathogens (Rothrock, C. S., and Gottlieb, D. 1981. Importance of antibiotic production in antagonism of selected Streptomyces species to two soil-borne plant pathogens. Journal of Antibiotics (Tokyo), 34 (7): 830-835).

A series of medium optimisation tests were conducted to determine which medium was most suitable for enhancing production of the bioactive compound. The strain only produced the bioactive compound when grown on solid medium. It is not produced in liquid culture. It was found that Medium I was most effective production medium. The recipe for Medium I has been previously published (Kepplinger B. The discovery and characterisation of novel antibiotics fmm Amycolatopsls isolate DEM30355. EngD Thesis, University of Newcastle upon Tyne, 2016) and is as follows: Agar (Melford™ Labs) (10 g/1), Soluble Starch (Sigma™) 20 g/1, Meat Extract (Sigma™) 2 g/1, Yeast Extract (Melford™ Labs), 2 g/1, Glucose (Fisher Scientific™), 10 g/l, Casein Hydrolysate, 4 g/l, Calcium Carbonate, (Melford™ Labs), 3 g/1. distilled water to 1 litre then pH to pH7.

### Purification of the Zinc Complex of the Invention

DEM21859 was inoculated onto the Medium I plates in the form of spores prepared by the method as described in Kieser T, Bibb M J, Buttner M J, Chater K F and Hopwood D A. “Practical Streptomyces Genetics” 2000, The John Innes Foundation, Norwich. The spores were are streaked evenly over the surface to produce a confluent lawn and after 2-3 days growth at 25-30° C. the plates were bioassayed against S. pombe using the “Plug test” bioassay method as described in the “Materials and Methods” of Lewis et al., 2017. When production of the compound was satisfactory (as determined by halo size and pombe phenotype induced) the plates were harvested. The agar/cells from the plates were harvested by firstly being roughly chopped up with a spatula and then disrupted by being forced through a 50 ml syringe into a plastic bag. The bagged agar/cell mass was then flattened out and frozen to −80° C.

The agar/cell mass was processed in 1 litre (˜40 plates) batches. A pack of agar/cell mass derived from 1 litre of plates was removed from the freezer and broken up to form chunks which were placed in a plastic bag which was immersed in warm water and allowed to thaw. The thawed agar/cell slurry was then squeezed through a cheesecloth, with the crush liquid (˜500 ml) passing through, whilst the agar/cell residue retained in the cloth was discarded.

The crush liquid was then extracted five times, each time with a volume of ethyl acetate equal to the volume of crush liquid. The five 500 ml portions (i.e. 2.5 litres in total) of ethyl acetate were then pooled and rotary evaporated to dryness using a standard Büchi™ rotary evaporator. The dried material was stored in a flask in a nitrogen atmosphere at −80° C. until required. [51] A large-scale bulk purification used 24 litres of agar/cell material prepared as described above in paragraphs [0481]-[050]. The dried material was removed from the freezer and split into three portions for easy handling. Each portion was dissolved in 25 ml methanol which was then added to 475 ml of water. The resultant three 500 ml portions were then each extracted three times with ˜300 ml dichloromethane (DCM). The bioactive compound is soluble in DCM. The ˜2,700 ml of DCM extracts were then pooled, and after 30 ml (˜15g) of silica powder was added, the mixture was evaporated to dryness using a standard Büchi™ rotary evaporator.

The silica powder with the S149 compound adsorbed onto it was divided into two equal portions of ˜7.5 g and each portion was used in normal phase “flash” chromatography using a Biotage™ Isolera™ machine and a Biotage™ KP-Sil 50 g column. A chloroform-methanol gradient was used to elute the bound material from the silica in which the percentage of methanol went from 0%-100%. No formic acid was added to the solvents as this degrades the bioactive compound. The eluate from the column was fractionated and the presence of the bioactive compound (S149) determined using the “Filter disc assay” method as described in the “Materials and Methods” of Lewis et al., 2017. The active fractions eluted from the two normal phase chromatography columns were pooled, and after removal of the solvent using a GeneVac™ Series U system equipped with a GeneVac™ VC3000TA condenser unit, the dried material was resuspended in 5 ml of methanol which was loaded onto a size exclusion column. The column was run at a flow rate of 1 ml/min with 1,200 ml of methanol and the eluate fractionated. The bioactive fractions, as determined using the “Filter disc assay” method as described in the “Materials and Methods” of Lewis et al., 2017, were pooled to give ˜15 ml of material which was diluted with 60 ml of water to give 75 ml of a 20% methanol solution. This was used in reverse phase chromatography using a Biotage™ Isolera™ chromatography machine in which the bioactive material was loaded by injection onto a C18 SNAP Ultra 12 g Biotage™ column and eluted using an acetonitrile-water gradient where the percentage of acetonitrile was increased from 20%-100%.

The bioactive fractions (˜30 ml in total), identified using the “Filter disc assay” method as described in the “Materials and Methods” of Lewis et al., 2017, were pooled, and after removal of the solvent using a GeneVac™ Series II system equipped with a GeneVac™ VC3000TA condenser unit, the dried material was resuspended in 20% methanol. The material was analysed using an Agilent™ Technologies 1260 Infinity liquid chromatography machine equipped with an Agilent™ 150×4.6 mm Eclipse Plus™ C18 3.51 μm reverse-phase column and a Hichrom™ C18 guard column. The material was eluted from the column using an acidified (0.1% formic acid) water-acetonitrile gradient, where the percentage of acetonitrile was increased from 20%-100% over 30 min, after which it was decreased to 20% over 1 min and finally the column was washed for 4 min using a 20% acetonitrile, 80% water mix. A flow rate of 1 ml/min was used, monitoring with a DAD array at λ==210, 254, 273, 280, 300 and 600 nm relative to λ=360 nm.

An example of the u.v. absorbance pattern (λ=254 nm) is shown in FIG. 1 and indicates the presence of four peaks labelled 0-3. An integrated fraction collector was used to peak-pick material and collect column eluate corresponding to the peaks which were then bioassayed using the “Filter disc assay” method as described in the “Materials and Methods” of Lewis el al., 2017. The results indicated that the material in Peaks 1-3 was more strongly active than the material in Peak 0. The haloes in the S. pombe lawn around discs impregnated with material from Peaks 1-3 developed after 24 hours incubation and were very distinct, whereas the haloes around discs impregnated with material from Peak 0 took ˜48 hours to develop and were more indistinct, indicating the bioactivity of the Peak 0 derived material was lower than the bioactivity of the material from the other peaks.

### Analysis and Identification of the Zinc Complex of the Invention

Material from Peaks 0-3 was collected by “peak-picking” and/or “time-slicing” using the Agilent™ Technologies 1260 Infinity liquid chromatography machine and sent for direct injection electrospray mass spectrometry (ESI-MS) using a LTQ-FT (Thenmo™) mass spectrometer with a 7T magnet at the Pinnacle Laboratory at The University of Newcastle. The results (FIG. 2) indicated that Peaks 1-3 contained an apparently identical compound, Peak 1 of which produced an ion of m/z=745.2735 [M+H⁺] and of accurate mass of 744.266. A sodium adduct (Peak 1: m/z=767.2551 [M+Na⁺]), is also present for all three MS spectra.

Examination of the MS-MS spectra (FIG. 3) of Peaks 1-3 indicated they are all are very similar, with all three spectra possessing the same ions. It was concluded that all three compounds possess the same internal arrangement of atoms as well as the same overall mass. The differing elution times of Peaks 1-3 from the HPLC column are thus probably due to each peak representing a differently charged form of the same molecule, rather than them being different molecules per se.

The material from Peak 0 was also collected by “peak-picking” using the Agilent™ Technologies 1260 Infinity liquid chromatography machine integrated fraction collector and sent for direct injection mass spectrometry. The results are shown in FIG. 4.

The results (FIG. 4A) obtained from the mass spectrometry of Peak 0 derived material indicated that it contains a mixture of ions with the two most abundant being m/z=683.3614 [M+H⁺] and m/z=653.3655 [M+H⁺], in addition to the minor ion at m/z=745.2755 [M+H⁺]. It is thought that the m/z=745.2755 [M+H⁺] species is the same as those already seen in Peaks 1-3 (m/z=745.2735 [M+H⁺] for Peak 1 shown in FIG. 2A).

Interpretation of this data was assisted by consideration of the MS-MS spectra obtained for the Peak 0 ions, (FIGS. 4B & FIG. 4C). The MS-MS data (FIG. 4B) indicated that the m/z=745.2755 [M+H⁺] ion generates a m/z=682.8996 [M+H⁺] species, which when the slight inaccuracy inherent in MS-MS analysis is allowed for is identical to the m/z=683.3614 [M+H⁺] species observed in the MS data (FIG. 4A). The MS-MS data for the m/z=683.3614 [M+H⁺] ion (FIG. 4C) indicated that it generates a m/z=652.9787 [M+H⁺] species, which when the slight inaccuracy inherent in MS-MS analysis is allowed for is identical to the m/z=653.3655 [M+H⁺] species observed in the MS data (FIG. 4A). We interpret this as the m/z=683.3614 [M+H⁺] species loses a nitroso group (—N═O) when it forms the m/z=653.3655 [M+H⁺] species, as the mass difference correlates with the mass of this group.

We also note that the ˜m/z=745.2735 [M+H⁺] species seen in Peaks 1-3 (FIG. 2) and Peak 0 (FIG. 4A) possesses a distinctive isotope pattern which is not seen with the m/z=683.3614 [M+H⁺] species. We interpret these data as the ˜m/z=745.2762 [M+H⁺] as being a Zn²⁺ complexed form and the m/z=683.3614 [M+H⁺] species representing the non- Zn²⁺ complexed form. The mass difference between the m/z=745.2755 [M+H⁺] and m/z=683.3614 [M+H⁺] species is 61.9141. This figure is consistent with the loss of a Zn²⁺ (mass=63.929) from the complexed form (m/z=745.2755 [M+H⁺]), and its consequent uptake of two H⁴ (so as to maintain charge neutrality) to give the non- Zn²⁺+ complexed form (m/z=683.3614 [M+H⁺]). The isotope pattern is consistent with the abundances of the natural isotopes of zinc as the results of a MS modelling study (FIG. 5B) based on an ion of formula C₃₁H₄₈N₆O₁₁ZnH⁺ closely correlate with the actual data (FIG. 5A) for the m/z=745.2735 [M+H⁺] ion, i.e. the Zn²⁺ complexed species obtained from Peak 1 (FIG. 2A).

S149 structural determination

The S149 molecule was purified as described above in paragraphs [048]-[052] i.e. by ethyl acetate extraction, followed by DCM extraction, followed by normal phase flash chromatography, followed by size exclusion chromatography and then reverse phase flash chromatography. As a final step the material from Peak 1 was “peak picked” multiple times (˜49 X) using the Agilent™ Technologies 1260 Infinity liquid chromatography machine integrated fraction collector as described in paragraph [053].

When dried down in a GeneVac™ the collected column eluate yielded 10-15 mg of compound. This material was supplied to Medina™ Ltd (Fundacibn MEDINA, Avda del Conocimiento 34, Parque Tecnológico Ciencias de la Salud, 18016, Armilla, Granada, Spain) and was analysed by mass spectrometry, and subsequently by NMR. The mass spectrometry results indicated that the mass obtained was consistent with predicted formulae of either C₃₀H₅₂N₂O₁₅Zn or C₃₁H₄₈N₆O₁₁Zn and this taken together with the 1H NMR data which provided details of the carbon skeleton of S149 indicated that S149 is a zinc complexed form of the known molecule JBIR-141, which has been published (Kawahara T, Kagaya N, Masuda Y, Doi T, Izumikawa M, Ohta K, Hirao A and Shin-ya K. 2015. Foxo3a inhibitors of microbial origin, JBIR-141 and JBIR-142.Organic Letters, 17(21): 5476-9). JBIR-141 is made by an actinomycete strain whose closest 16S rRNA match (99.9% sequence identity over 1483 bp) is Streptomyces panayensis, although another close match (98.8%) was Streptomyces sampsonii. The identification of S149 as a zinc complex of JBIR-141 is supported by the fact that the structure possesses a nitroso group whose loss was observed in the ESI-MS (see FIG. 4 and paragraph [059]). It should also be noted that the characteristic triple-peak absorbance spectrum of S149 (see FIG. 6) is consistent with the identification of S149 as a zinc complexed form of JBIR-141 as the tetramic acid moiety of S149, also present in JBIR-141, is largely responsible for this pattern.

The structure of JBIR-141 is shown in below, and in FIG. 7. This is also the structure of S149, with the exception that two hydrogen atoms are substituted by a zinc ion (Zn²⁺).

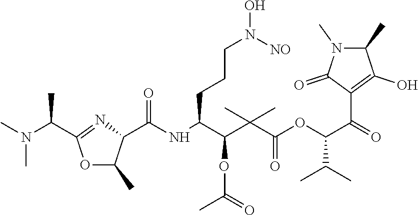

Kawahara et W., 2015 also describe a second molecule—JBIR-142 which is almost identical to JBIR-141 and differs from it only in that a hydrogen atom is substituted by a hydroxy group. The structure of JBIR-142 is as follows:

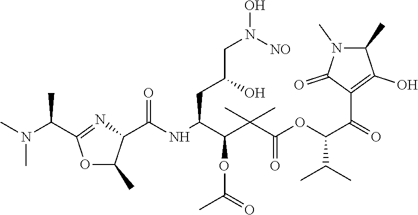

### Investigation of the Bioactivity of S149

The journal paper published by Kawahara et al.,—“Foxo3a inhibitors of microbial origin, JBIR-141 and JBIR-142” makes no reference to either JBIR-141 or JBIR-142 (or any of their variants/derivatives/degradation products etc) as being able to, or being capable of binding Zn²⁺. Thus, S149, i.e. the zinc complexed form of JBIR-141, represents a novel entity and is not anticipated by the disclosure of Kawahara et at, 2015.

Kawahara et al., show that both JBIR-141 & JBIR-142 possess the ability to inhibit the transcriptional activity of the forkhead transcription factor, Foxo3a. Although we have not produced the zinc complexed form of JBIR-142 in view of the fact that its published activity (Kawahara et al., 2015) is very similar to that of JBIR-141 and the difference in structure and chemical formula is a minor one we believe that the zinc complex of JBIR-142 possesses similar properties to that of S149 (the zinc complex of JBIR-141). Thus, the present invention also relates to, and encompasses, the zinc complex of JBIR-142.

We have shown that the zinc-free form of S149 (which is identical to JBIR-141) is not as bioactive as the zinc complexed form (S149) as the haloes/zones of inhibition in the S. pombe lawn surrounding the discs containing samples of the material peak-picked from Peak 0 took a much longer time to develop compared to the haloes surrounding discs containing material derived from Peaks 1-3. Prima facie, this shows that the zinc complexed form of JBIR-141, S149, has improved bioactivity over JBIR-141 and represents a significant improvement over the prior art.

To investigate the anticancer properties of S149 a number of different leukaemia cell lines were treated with S149 and its effects on their proliferation observed. Several acute myeloid leukaemia (AML) cell lines were used including Kasumi-1 and SKNO-1 (both t(8:21) rearranged AML cell lines), THP-1 and MV-411 (MLL rearranged cell lines) as well as HL-60 and OCI-AML3 (other AML cell lines). Additionally, two Burkitts Lymphoma (Non-Hodgkins Lymphoma) cell lines were used, Ramos and BL-41, as it has previously shown that knockdown of FOXO TF in these cell lines inhibits their proliferation (Gehringer F, Weissinger S E, Swier LJYM, Möller P, Wirth T, Ushmorov A. 2019. FOXO1 Confers Maintenance of the Dark Zone Proliferation and Survival Program and Can Be Pharmacologically Targeted in Burkitt Lymphoma. Cancers, 2019, 11: 1427). Mesenchymal stromal cells (MSC) were used as a negative control.

All cell lines were cultured as described previously (Martinez-Soria N, McKenzie L, Draper J, Ptasinska A, Issa H, Potluri S, Blair H J, Pickin A, Isa A, Suyin-Chin P, Tirtakusuma R, et al., 2018. The oncogenic transcription factor RUNXI/E7O corrupts cell cycle regulation to drive leukemic transformation. Cancer Cell, 34: 626-642; Gehringer F, Weissinger S E, Swier LJYM, Miller P, Wirth T, Ushmorov A. 2019. FOXOI Confers Maintenance of the Dark Zone Pmliferation and Survival Program and Can Be Pharmacologically Targeted in Burkett Lymphoma. Cancers, 11: 1427). The effect of S149 on proliferation of the cell lines was determined as described previously (Scherr M, Kirchoff H, Battmer K, Wohlan K, Lee C-W, Ricke-Hoch M, Erschow S, Law E, Kloos A, Heuser M et al., 2019. Optimized induction of mitochondria) apoptosis for chemotherapy free treatment of BCR-ABL+ acute lymphoblactic leukemia. Leukemia, 33: 1313-1323). Briefly, cells were plated in 96 well plates at 1×10⁵ cells/ml in the presence of increasing concentrations of S149 (0.1, 1.0, 10, 100, 1000 & 10,000 nM) dissolved in ethanol. Each cell line was tested in triplicate, i.e. 3 wells per cell line per S149 concentration. Negative controls using ethanol only were incorporated in the assay. The number of viable cells was determined 72 hours later using CyQuant™ Cell proliferation Assay, the fluorescence being read using an ELISA plate reader. The data are represented as a mean f SD of three independent replicates per cell-line, per S149 concentration, expressed as a percentage of the results obtained for the corresponding ethanol only negative control. EC₅₀ values were calculated using GraphPad Prism™ software.

The results are presented in FIGS. 8-16 which show the dose response curves obtained. The data clearly show that the proliferation of the cancer cell lines is significantly impaired by S149 whereas the proliferation of the MSC is far less inhibited. The EC₅₀ values (FIG. 17) correlate with these data and show that S149 inhibits the proliferation of the cancer cell lines 100-2,000 X more than it does that of the MSC.

Exposure to zinc ions has been shown to modulate FoxO signalling in human hepatoma cells as Zn²⁺ activates the AKT pathway leading to phosphorylation of FoxO factors and their export from the nucleus to the cytoplasm where they are inactive (Walter P L, Kampotter A, Eckers A, Barthel A, Schmoll D, Sies H, and Klotz L-O. 2006. Modulation of FoxO signaling in human hepatoma cells by exposure to copper or zinc ions. Archives of Biochemistry and Biophysics, 454: 107-113. It has also been shown that zinc ions (100 μM zinc acetate) are necessary for β-thujaplicin to induce phosphorylation (and consequent nuclear export and inactivation) of FOXO 1a (Cameron A R, Anil S, Sutherland E, Harthill J, and Rena G. 2010. Zinc-dependant effscts of small molecules on the insulin sensitive transcription factor FOXOJa and gluconeogenic genes. Metallomics, (2): 195-203).

These papers initially suggested to us the possibility that S149 is mediating its effects by increasing the supply of intracellular zinc ions. However, we believe, based on the EC₅₀ data for S149 (FIG. 17) which indicate it is effective at extremely low concentrations (nM) that the effect of 5149 is more likely to be a specific interaction with a target molecule, rather than a non specific mechanism related to it increasing the supply of Zn²⁺.

We also note that the small-cell phenotype observed in S. pombe on treatment with S149 may be explained by it inhibiting a forkhead transcription factor, in this case Fkh2 whose deletion has been shown to induce a range of abnormal cell phenotypes including “abnormally small and round” cells (see FIG. 2 of Bulmer R, Pic-Taylor A, Whitehall S K, Martin K A, Millar J B A, Quinn J and Morgan B A. 2004. The forkhead transcription factor Fkh2 regulates the cell division cycle of Schizosaccharomyces pombe. Eukaryotic Cell, 3(4): 944-954).

All references mentioned in this document are to be considered to be incorporated herein in their entirety. While specific embodiments of the invention have been described herein for the purpose of reference and illustration, various modifications will be apparent to a person skilled in the art without departing from the scope of the invention as defined by the appended claims.

### FIGURE LEGENDS

FIG. 1: HPLC trace (λ=254 nm) of a purified sample of S149. The four peaks obtained, —(Peaks 0-3) are labelled above by numbers.

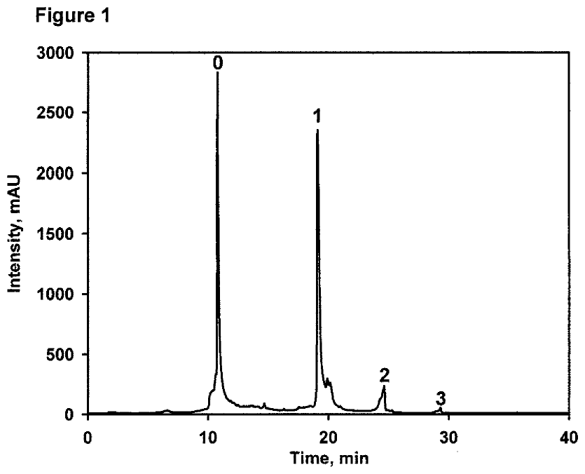

FIG. 2: Mass spectrometry data obtained when material from Peak 1 (Panel A); Peak 2 (Panel B) and Peak 3 (Panel C) was analysed. The masses of the major ions are shown above the relevant peaks.

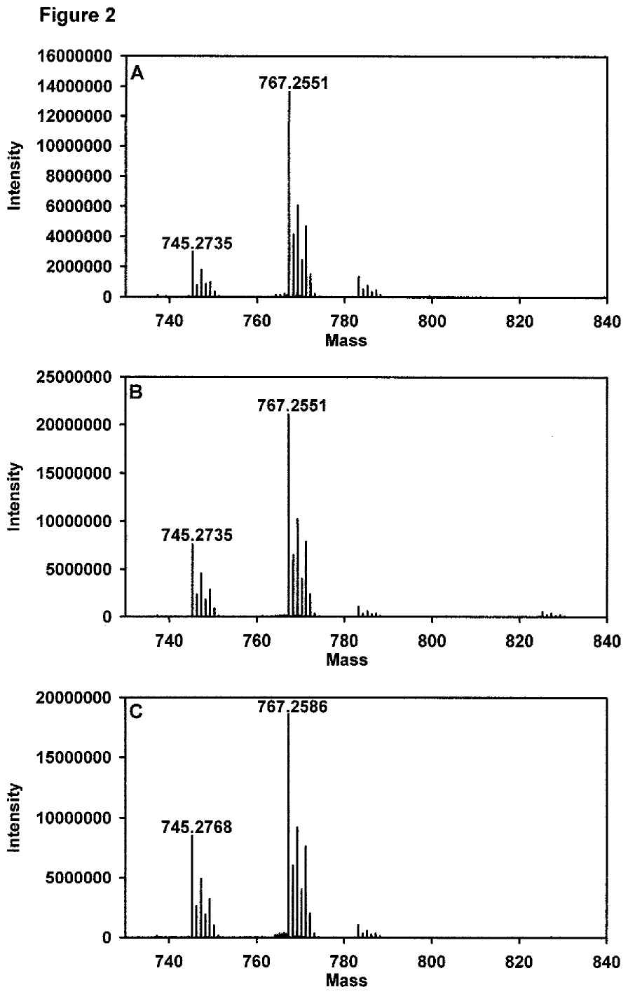

FIG. 3: MS-MS mass spectrometry data obtained when the ˜m/z=745.27 [M+H⁺] ion from Peak 1 (Panel A); Peak 2 (Panel B) and Peak 3 (Panel C) was analysed. The masses of the major ions are shown above the relevant peaks.

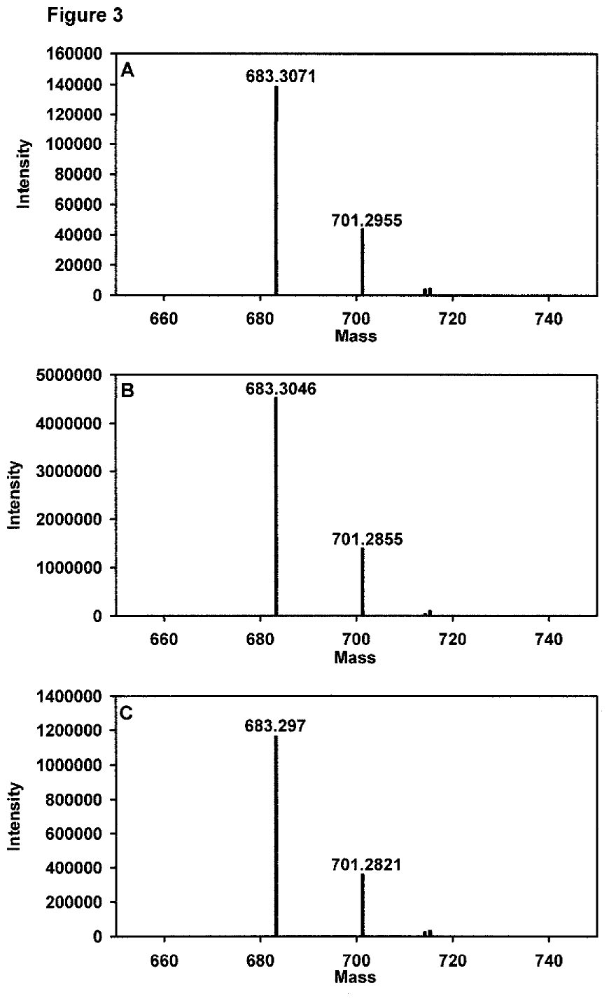

FIG. 4: Mass spectrometry data obtained when material from Peak 0 was analysed (Panel A); MS-MS data obtained when the ˜m/z=745.2755 [M+H⁺] ion from Peak 0 (Panel B) was analysed; MS-MS data obtained when the ˜m/z=683.3614 [M+H⁺] ion from Peak 0 (Panel C) was analysed. The masses of the major ions are shown above the relevant peaks.

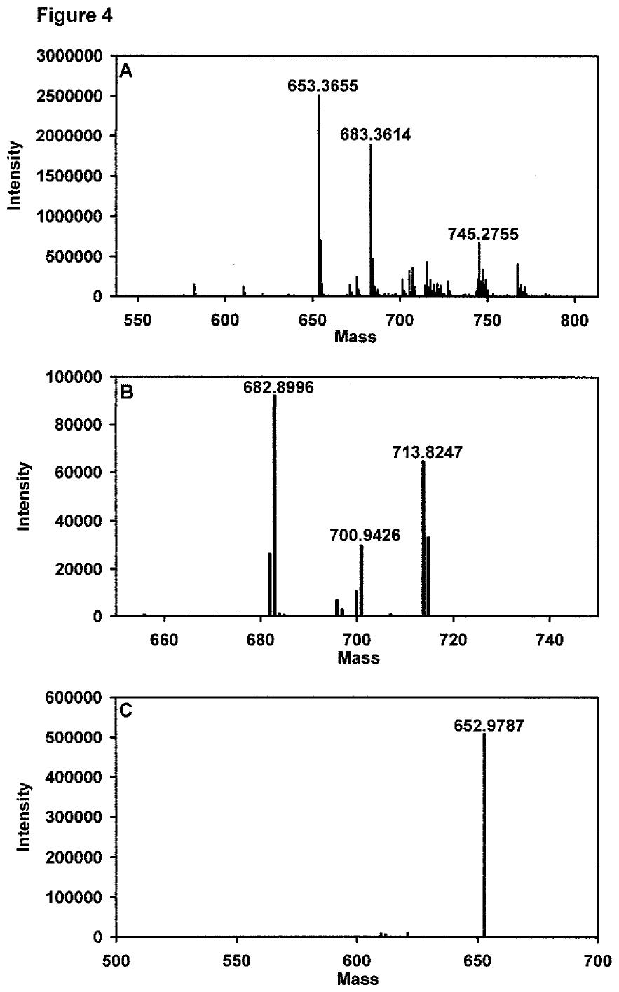

FIG. 5: Mass spectrometry data illustrating the isotope profile of the m/z=745.2735 [M+H⁺] ion (Panel A) and modelling data for the ion with the predicted formula C₃₁H₄₈N₆O₁₁ZnH⁺(Panel B).

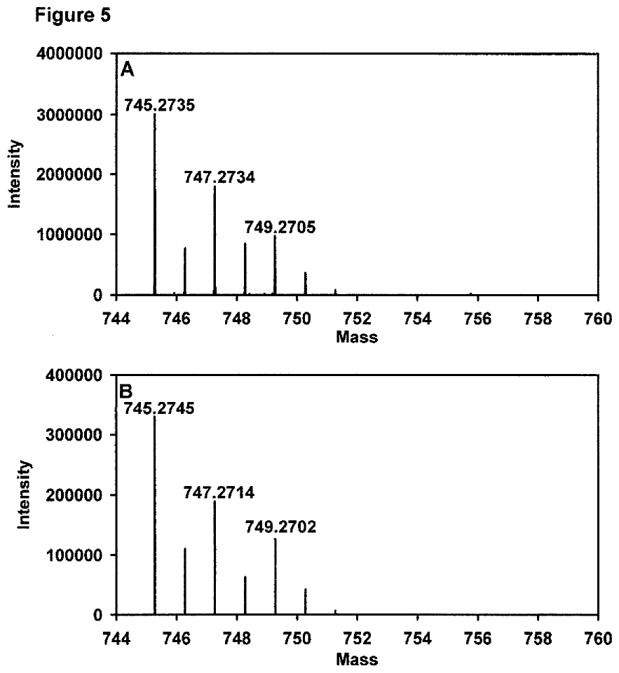

FIG. 6: HPLC Absorbance spectra data obtained when material from Peak 0 (Panel A); Peak 1 (Panel B) and Peak 2 (Panel C) was analysed.

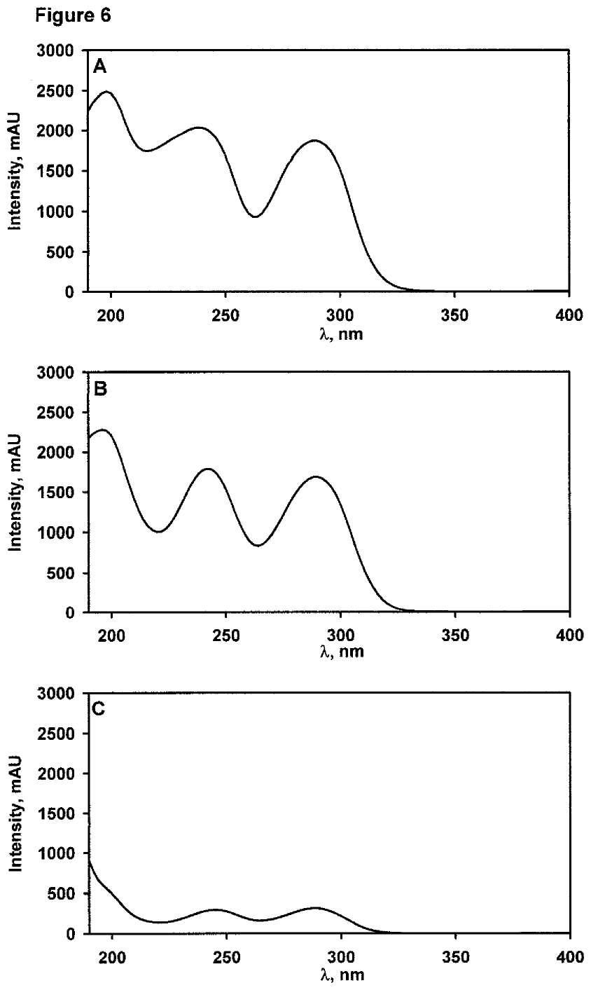

FIG. 7: Diagram illustrating the structure of JBIR-141. This is also the structure of the zinc-free form of S149 (as determined by Medina™ Ltd) and is the bioactive entity from Peak 0.

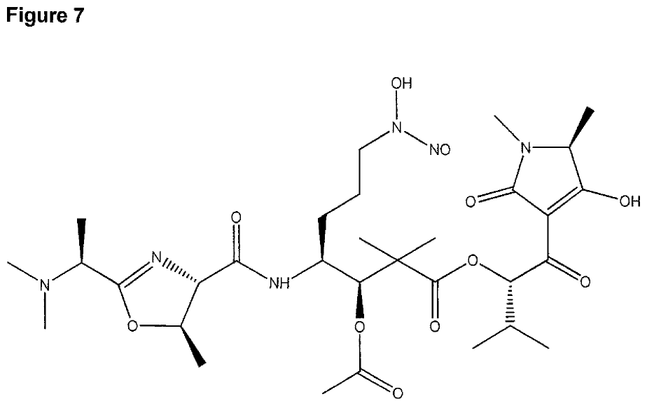

FIG. 8: Dose response data obtained after 72 hr treatment of MSC with S149.

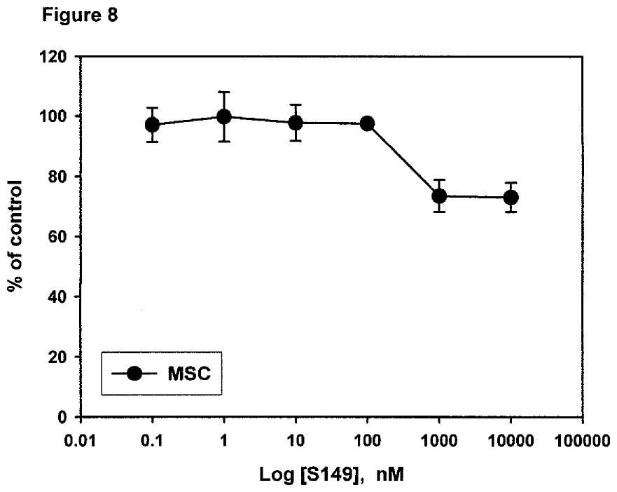

FIG. 9: Dose response data obtained after 72 hr treatment of Kasumi-1 with S149.

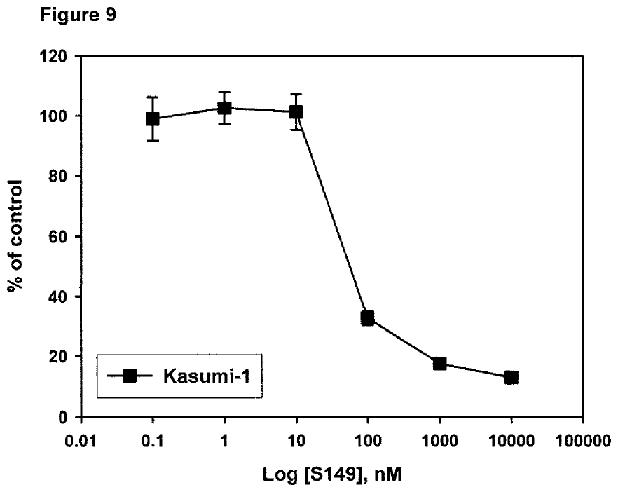

FIG. 10: Dose response data obtained after 72 hr treatment of SKNO-1 with S149.

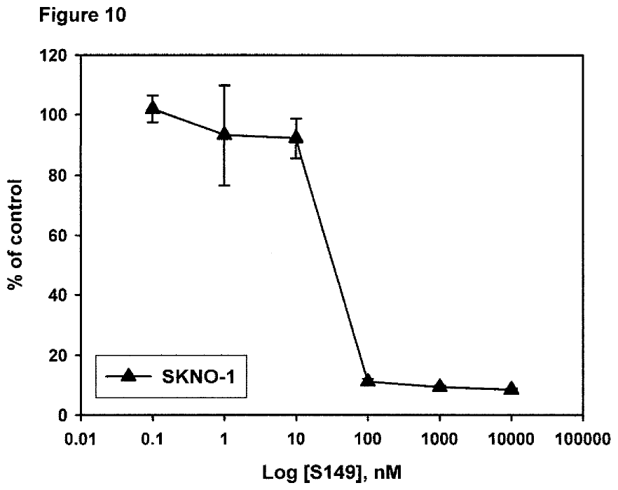

FIG. 11: Dose response data obtained after 72 hr treatment of HL-60 with S149.

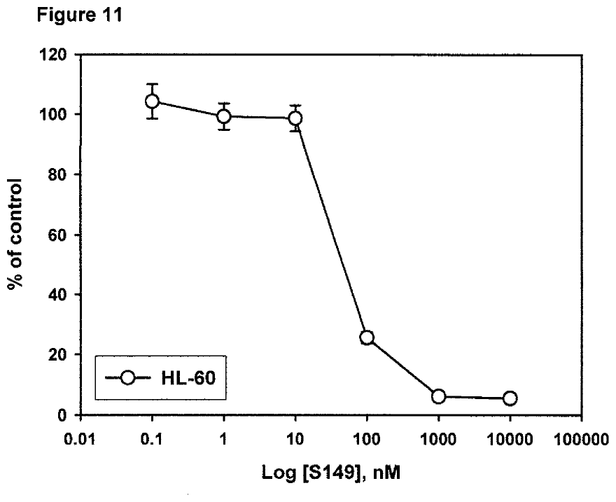

FIG. 12: Dose response data obtained after 72 hr treatment of OCI-AML3 with S149.

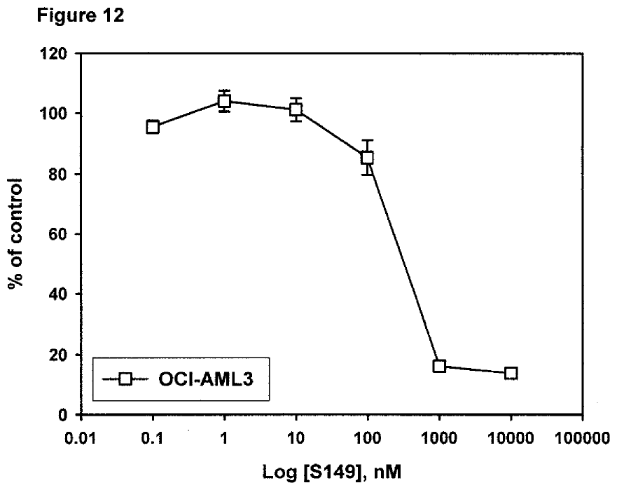

FIG. 13: Dose response data obtained after 72 hr treatment of THP-1 with S149.

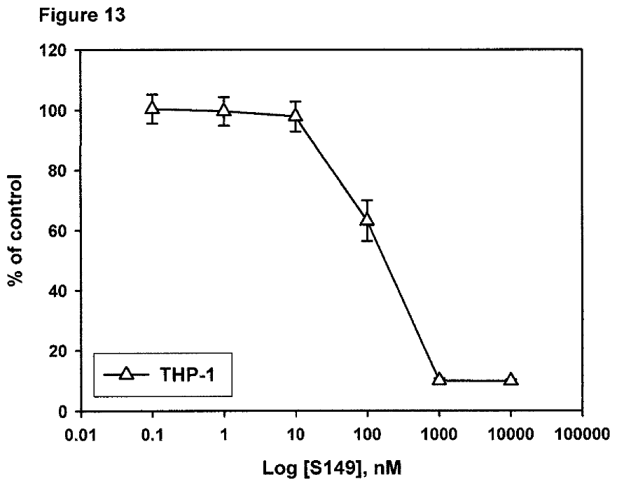

FIG. 14: Dose response data obtained after 72 hr treatment of MV-4-11 with S149.

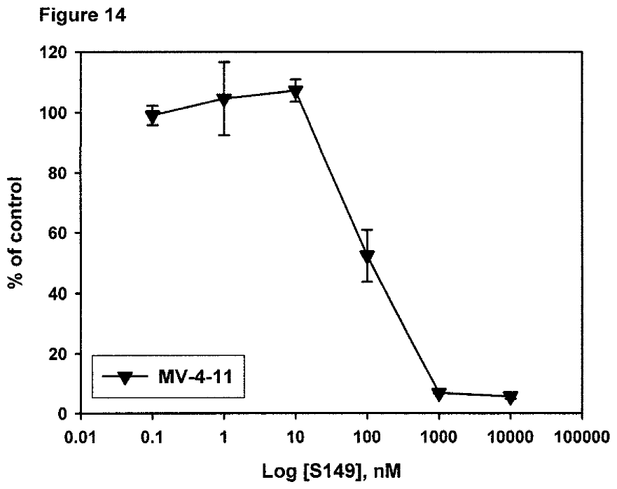

FIG. 15: Dose response data obtained after 72 hr treatment of Ramos with S149.

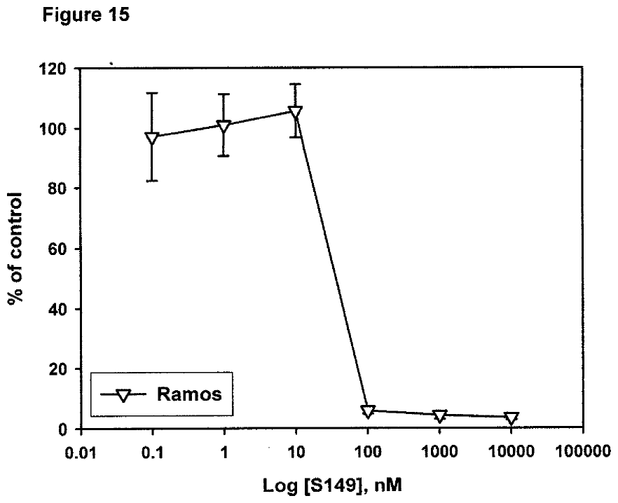

FIG. 16: Dose response data obtained after 72 hr treatment of BL-41 with S149.

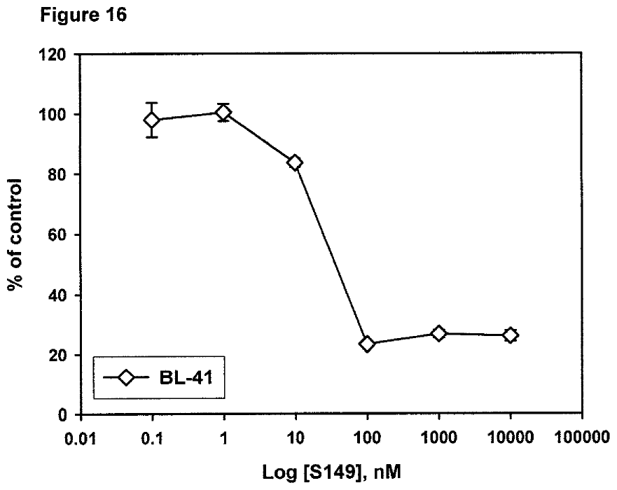

FIG. 17: Table providing S149 ECK, data for leukaemia cell lines and MSC control.

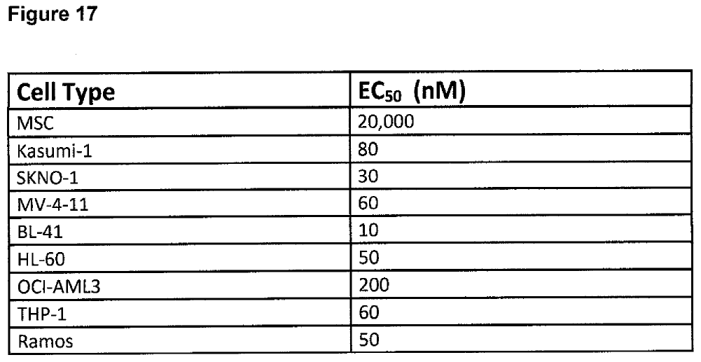

1: A zinc complex of a compound which has the chemical structure: 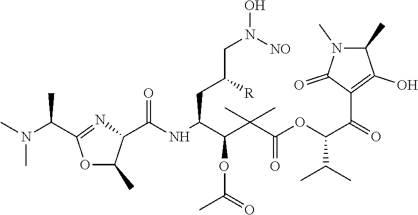
where R═H and the compound is known as JBIR-141, or where RAH and the compound is known as JBIR-142, and the zinc complex is either a complex of JBIR-141 and a zinc ion and has the chemical formula C₃₁H₄₈N₆O₁₁Zn, or is a complex of JBIR-142 and a zinc ion and has the chemical formula C₃₁H₄₈N₆O₁₂Zn.

2: A zinc complex as defined in claim 1 for use as a medicament.

3: A zinc complex as defined in claim 1 for use in the treatment of diseases associated with the overexpression of FoxO transcription factors.

4: A zinc complex as defined in claim 1 for use in the treatment of diseases associated with the overexpression and predominantly nuclear localisation of FoxO transcription factors.

5: A zinc complex as defined in claim 1 for use in the treatment of cancer associated with the overexpression of FoxO transcription factors.

6: A zinc complex as defined in claim 1 for use in the treatment of cancer associated with the overexpression and predominantly nuclear localisation of FoxO transcription factors.

7: A zinc complex as defined in claim 1 for use in the treatment of acute or chronic myeloid leukaemia.

8: A pharmaceutical composition comprising a zinc complex according to claim 1 or a pharmaceutically acceptable salt or solvate thereof, and one or more pharmaceutically acceptable excipients or carriers.
